(19) United States
(12) Patent Application Publication
(10) Pub. No.: US 2025/0114532 A1
(43) Pub. Date: Apr. 10, 2025

(54) Sub-assembly for a medicament delivery device

(71) Applicant: SHL Medical AG, Zug (CH)

(72) Inventor: Jason Mondro, Deerfield Beach, FL (US)

(21) Appl. No.: 18/729,948

(22) PCT Filed: Jan. 9, 2023
(86) PCT No.: PCT/EP2023/050280

§ 371 Date: Jul. 18, 2024

Related U.S. Application Data
(60) Provisional application No. 63/301,575, filed on Jan. 21, 2022.

(30) Foreign Application Priority Data
Feb. 2, 2022 (EP) 22154695.5

(51) Int. Cl. A61M 5/315; A61M 5/20

(52) U.S. Cl. CPC A61M 5/31511; A61M 5/20

(57) ABSTRACT

The present disclosure provides a sub-assembly for a medicament delivery device, the sub-assembly extending along a longitudinal axis in a longitudinal direction from a proximal end to a distal end. The sub-assembly includes a plunger rod, the plunger rod comprising a proximally facing surface that faces towards the proximal end. The sub-assembly further includes a feedback component comprising a distally facing surface facing towards the distal end, so that when the plunger rod moves towards the proximal end during use of the medicament delivery device, the distally facing surface of the feedback component is engaged by the proximally facing surface of the plunger rod, is subsequently moved towards the proximal end by the plunger rod, and is finally disengaged from the proximally facing surface of the plunger rod, wherein the feedback component comprises a spring.

### CROSS-REFERENCE TO RELATED APPLICATIONS

The present application is a U.S. National Phase Application pursuant to 35 U.S.C. § 371 of International Application No. PCT/EP2023/050280 filed Jan. 9, 2023, which claims priority to (i) U.S. Provisional Patent Application No. 63/301,575 filed Jan. 21, 2022, and (ii) European Patent Application No. 22154695.5 filed Feb. 2, 2022. The entire disclosure contents of these applications are herewith incorporated by reference into the present application.

### TECHNICAL FIELD

The invention concerns sub-assemblies for medicament delivery devices, and particularly sub-assemblies comprising feedback components.

### BACKGROUND

Some medicament delivery devices provide feedback to their users during operation, such as with the U-bracket described in WO2011/123024. While such systems can provide effective feedback, the applicant has appreciated that an alternative feedback mechanism could usefully be provided, potentially providing various advantages when compared to existing feedback mechanisms.

### SUMMARY

Reference should now be made to the appended claims.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

An aspect concerns a sub-assembly for a medicament delivery device, the sub-assembly extending along a longitudinal axis in a longitudinal direction from a proximal end to a distal end, the sub-assembly comprising: a plunger rod, the plunger rod comprising a proximally facing surface that faces towards the proximal end; and a feedback component comprising a distally facing surface facing towards the distal end, so that when the plunger rod moves towards the proximal end during use of the medicament delivery device, the distally facing surface of the feedback component is engaged by the proximally facing surface of the plunger rod, is subsequently moved towards the proximal end by the plunger rod, and is finally disengaged from the proximally facing surface of the plunger rod, wherein the feedback component comprises a spring. This can enable the removal of a metal component (i.e. removal of the U-bracket), thereby potentially reducing cost. In addition to being able to provide feedback, this structure can also provide tolerance management, thereby providing two functions from a single structure, which can improve simplicity and cost.

Optionally, the plunger rod comprises a protrusion, and the proximally facing surface of the plunger rod is on the protrusion. Optionally, the protrusion extends from an outer surface of the plunger rod. Optionally, the plunger rod comprises a flexible arm, and the protrusion is attached to the flexible arm. Optionally, the protrusion is attached to a distal end of the flexible arm. Optionally, the flexible arm extends in the longitudinal direction, and the flexible arm is attached to the rest of the plunger rod at a proximal end of the flexible arm. Optionally, the proximally facing surface of the plunger rod is angled away from the longitudinal axis.

Optionally, the sub-assembly comprises a power source for moving the plunger rod towards the proximal end. Optionally, the power source is a spring. Optionally, the spring is inside the plunger rod. Optionally, the sub-assembly comprises a housing, and the plunger rod is at least partly inside the housing.

Optionally, the sub-assembly comprises a proximally facing surface of a first further component, and wherein the distally facing surface of the feedback component is adjacent to (or more specifically abuts) the proximally facing surface of the first further component. Particularly in the case where the feedback component abuts the proximally facing surface of the first further component, this can help with tolerance management. Optionally, the first further component is a housing. Optionally, the sub-assembly comprises a powerpack comprising the plunger rod, and the first further component is a component of the powerpack other than the plunger rod.

Optionally, the sub-assembly is arranged so that, when the distally facing surface of the feedback component disengages from the proximally facing surface of the plunger rod, the distally facing surface of the feedback component moves towards the distal end, thereby impacting the proximally facing surface of the first further component to provide feedback.

Optionally, the feedback component comprises a proximally facing surface facing towards the proximal end. Optionally, the sub-assembly comprises a distally facing surface of a second further component, and wherein the proximally facing surface of the feedback component is arranged adjacent to (or more specifically abuts) the distally facing surface of the second further component. Particularly in the case where the feedback component abuts the distally facing surface of the second further component, this can help with tolerance management. Optionally, the second further component is a housing. Optionally, the second further component is a syringe carrier. Optionally, the second further component is a syringe.

Optionally, the proximally facing surface of the feedback component is arranged adjacent to the distally facing surface of the second further component so that the spring is compressed when the distally facing surface of the feedback component is moved towards the proximal end by the plunger rod.

Optionally, the feedback component provides a start click to provide an indication of the start of an injection and/or an end click to provide an indication of the end of an injection.

Optionally, the feedback component comprises a disc. Optionally, a proximally facing surface of the disc is adjacent to a distally facing surface of the spring.

Another aspect concerns a medicament delivery device comprising the sub-assembly of any previous claim. Optionally, the medicament delivery device is an autoinjector.

Another aspect concerns a method of providing feedback for a medicament delivery device, comprising the following steps in the following order: moving a plunger rod in the proximal direction relative to a feedback component to engage a distally facing surface of the feedback component with a proximally facing surface of the plunger rod; moving the plunger rod in the proximal direction while the distally facing surface of the feedback component and the proximally facing surface of the plunger rod are engaged, thereby moving the feedback component in the proximal direction along with the plunger rod; disengaging the distally facing surface of the feedback component from the proximally facing surface of the plunger rod, thereby releasing the feedback component so that the feedback component moves in the distal direction relative to the plunger rod and creates feedback. Optionally, the feedback is audible, tactile and/or visual.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIGS. 1 to 4 each show the same cross-section view of part of a medicament delivery device containing a sub-assembly as described herein at different stages during use of the medicament delivery device.

FIG. 5 shows a perspective view of an autoinjector that could contain the sub-assembly shown in FIGS. 1 to 4.

FIG. 6 shows a perspective view of the plunger rod shown in FIGS. 1 to 4.

FIG. 7 shows a perspective of a distal part of the plunger rod of FIG. 6.

FIG. 8 shows a perspective view of the feedback component shown in FIGS. 1 to 4.

### DETAILED DESCRIPTION

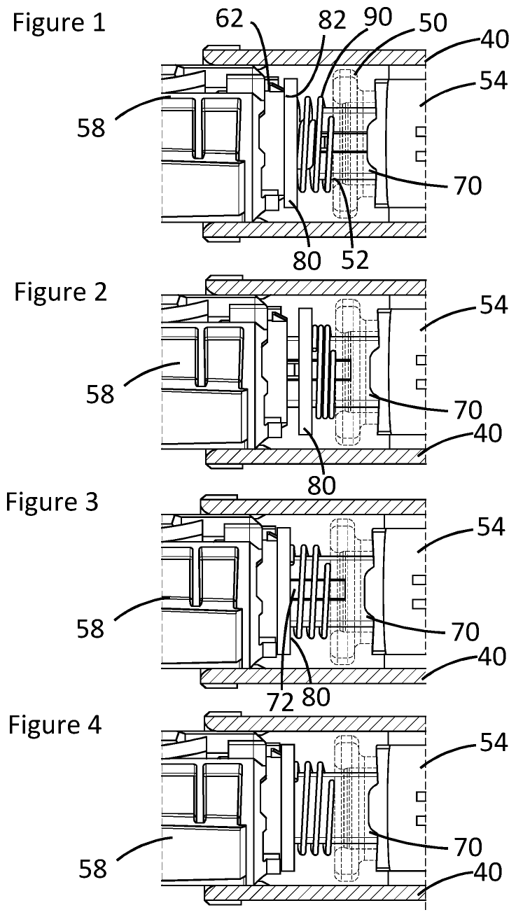

Sub-assemblies for a medicament delivery device will now be described, with the sub-assemblies comprising a feedback component. The feedback component illustrated in the figures comprises a disc 80 and a spring 90 (see in particular FIG. 8). In addition to the disc 80 and the spring 90, a needle guard 40 (medicament delivery member guard), a syringe 50, a syringe carrier 54, a rotator 58 and a plunger rod 70 are also visible in FIG. 1, along with a small part of a rear cap 62. A proximally facing surface of the spring 90 (which is at a proximal end of the spring 90 in this example) abuts a distally facing surface 52 of a syringe 50 (which is at a distal end of the syringe 50 in this example). A distally facing surface of the spring 90 (which is at a distal end of the spring 90 in the example) abuts a proximally facing surface of the disc 80 (which is at a distal end of the disc 80 in this example). A distally facing surface of the disc 80 (which is at a distal end of the disc 80 in this example) abuts a proximally facing surface of the rotator 58 (which is at a proximal end of the rotator 58 in this example).

The plunger rod 70 will now be described with particular reference to FIGS. 6 and 7. The plunger rod 70 comprises two flexible arms 72, with each arm being attached to the rest of the plunger rod at one end (in this example the proximal end of the arm) and with each arm comprising a protrusion 74 extending away from the axis 12 at the other end (in this example the distal end of the arm). As can be seen in FIG. 7 in particular, a proximally facing surface 76 of the protrusion 74 is angled away from the axis 12. Having the proximally facing surface 76 of the protrusion 74 angled away from the axis 12 (rather than the surface 76 extending in a direction parallel to the axis) is optional, but can help the protrusion slide past the distally facing surface of the feedback component (in this example the distally facing surface 82 of the disc 80) without needing the protrusion to flex. The angle at which the proximally facing surface 76 of the protrusion 74 is angled away from the axis could be varied, for example to vary the ease with which the protrusion can pass the feedback component, therefore varying the speed with which the feedback component impacts a further component (i.e. another component) to create feedback. The speed of impact would typically be larger for a smaller angle (with an angle of 0 degrees considered to provide a proximally facing surface 76 of the protrusion 74 where the surface 76 extends perpendicular to the axis). The angle could be between 0 and 80 degrees for example, or between 15 and 60 degrees, or between 30 and 45 degrees. The particular shape of the protrusion and of the corresponding part of the feedback component that the protrusion engages (the distally facing surface of the disc 80 in the example above) can also be varied to vary the friction between the feedback component and the protrusion, which can also be used to vary the speed of impact.

The mechanism of action of the illustrated example will now be described with reference to FIGS. 1 to 4, with the figures showing the action in reverse (i.e. FIG. 1 is the latest in time, and FIG. 4 is the earliest). FIG. 4 shows the relative positions of a number of components before the arms 72 of the plunger rod 70 engage the feedback component. The spring 90 is therefore in its extended state and the disc 80 is in its distal position.

When the plunger rod initially starts moving towards the proximal end of the device, there is initially no change in the state of the feedback component (as in this example the feedback component provides an end click). Part of the way through medicament delivery, the plunger rod will reach the state shown in FIG. 3, which shows the relative positions of the components when the arms 72 of the plunger rod 70 engage the feedback component (by engagement of the proximally facing surface 76 of the protrusion 74 with the distally facing surface 82 of the disc 80). In this example, where the feedback component provides an end click, this engagement would typically only happen late in the medicament delivery cycle, for example after at least 60% of the medicament has been delivered, or more particularly between 70 and 90% of the medicament has been delivered.

As the plunger rod continues to move towards the proximal end of the device, the plunger rod will now pull the feedback component (and particularly the disc 80) in the proximal direction, thereby compressing the spring 90 from its extended state to a compressed state. This is shown in FIG. 2, which shows the relative positions of the components when the distally facing surface 82 of the feedback component has been moved towards the proximal end by the plunger rod but before the distally facing surface 82 of the feedback component has been disengaged from the proximally facing surface 76 of the plunger rod 70.

The point at which the proximally facing surface 76 of the protrusion 74 of the arm 72 disengages from the distally facing surface 82 of the disc 80 can be varied by varying several factors, such as the friction between the surfaces 76, 82, the angle of the surface 76, the angle of the surface 82, and the stiffness of the arm 72.

Once the proximally facing surface 76 of the protrusion 74 of the arm 72 disengages from the distally facing surface 82, the spring 90 pushes the disc 80 back toward the distal end of the device, which results in the distally facing surface 82 of the disc 80 impacting the proximally facing surface of the rotator 58, thereby creating feedback. The feedback would typically be audible, though the sudden movement of parts relative to one another could also provide tactile feedback due to a shifting of the device, and could also provide visual feedback if part or all of the feedback component is made visible, for example by providing a window or a transparent portion in a housing of the device.

FIG. 1 shows the relative positions of the components after the distally facing surface 82 of the feedback component has been disengaged from the proximally facing surface 76 of the plunger rod 70 and has rebounded back to its original position.

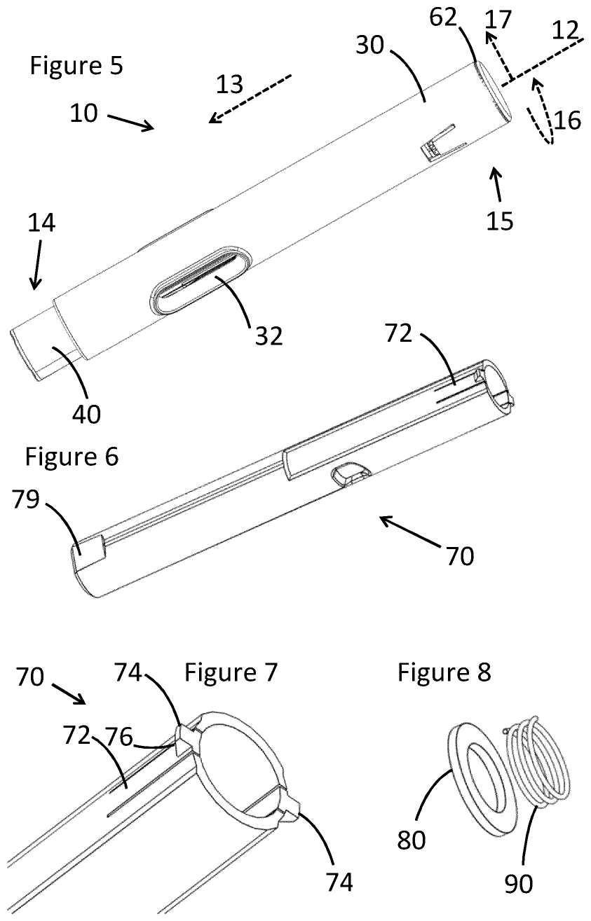

FIG. 5 shows an example of an autoinjector 10 that could include the sub-assembly as described above. A housing 30, along with an optional window 32 in the housing 30 and an optional needle guard 40, are visible. A small portion of a rear cap 62 is also visible. For reference, a longitudinal axis 12, a longitudinal direction 13, a proximal end 14, a distal end 15, a circumferential direction 16, and a radial direction 17 are shown.

A typical autoinjector would also include a powerpack to drive the plunger rod. The powerpack could provide power using a power source such as a spring, a battery or compressed gas, for example. The optional rear cap 62 can be part of the powerpack, and could house the power source. The syringe 50 would typically comprise a medicament delivery member, such as a needle or a jet injector. Although not essential, the powerpack would typically be locked until the device is activated for use, for example by the rotator 58 shown in the figures being rotated by the needle guard 40 (or more generally the medicament delivery member guard), or by another component such as a button. A medicament delivery container carrier such as a syringe carrier could also be provided, although the medicament delivery container could be supported by one or more other components instead, such as by a housing. Although an autoinjector is described, the sub-assembly could also be used in other injectors or other medicament delivery devices more generally, including non-automatic medicament delivery devices such as pen injectors, and components such as the rear cap, the powerpack, the rotator and the medicament delivery member guard are optional.

The sub-assembly would typically include a feedback component along with a plunger rod. Depending on the structure of the device, the sub-assembly can also include various other components with which the feedback component and the plunger rod interact.

Although not essential, a housing (e.g. the housing 30 and/or the rear cap 62) is typically provided to keep the components of the medicament delivery device together and/or to stop users from accidentally interfering with key device functionality. Instead of a syringe, other medicament delivery containers such as cartridges could be used.

In the example described above, the feedback component interacts with two other components in addition to interacting with the plunger rod 70, namely with the rotator 58 and the syringe 50. Instead of interacting with the rotator 58, the proximally facing surface (that interacts with a distally facing surface of the feedback component) could be provided by another component, for example a housing or a rear cap. The proximally facing surface could also be provided directly by the plunger rod—that is, a separate proximally facing surface beyond the one provided by the plunger rod is optional. This could particularly be the case in examples where the feedback is a start-of-injection feedback.

The interaction of the proximally facing surface of the feedback component with the syringe 50 can be beneficial as it can help support the syringe. Nevertheless, instead of being part of the syringe 50, the distally facing surface (that interacts with a proximally facing surface of the feedback component) could be provided by another component, for example a housing, a rear cap or a syringe carrier.

Although the surfaces of the feedback component that interact with other components are shown as being at the proximal end (the proximally facing surface of the spring) and the distal end (the distally facing surface of the disc) of the feedback component, the proximally facing surface could be spaced apart from the proximal end and/or the distally facing surface could be spaced apart from the distal end.

The example above is envisioned as providing feedback (an indication) that an injection is completed (end click). However, rearrangement of the components relative to one another could instead provide feedback at another time, for example before, at the start of or during an injection. For example, to provide feedback at the start of an injection, the arms 72 could be moved towards the proximal end of the plunger rod from the position shown in FIGS. 6 and 7. Optionally, more than one set of arms could be provided, for example with a first set of arms providing feedback at the start of an injection and a second set of arms providing feedback at the end of an injection, or with a plurality of sets of arms providing feedback during injection to provide so-called continuous feedback that an injection is in progress.

Two arms 72 opposite one another relative to the axis are shown in the figures; this structure can be beneficial as it can balance the forces within the device. However, one, three or more arms could be provided. Optionally, the arms are spaced out equally around the circumference of the plunger rod.

In the embodiment as depicted, it is envisioned that the primary means of feedback would be audible. However, the feedback could be audible, tactile and/or visual. The audible feedback is provided by parts hitting together, tactile feedback could be provided due to a shift in balance in the device (particularly if continuous feedback is provided), and visual feedback could be provided through a window in the housing in which the feedback component could disappear from a user's view and then reappear.

The particular structure of the feedback component as described above is also optional, and could be altered in various ways. For example, the spring is closer to the proximal end of the sub-assembly than the disc, but the spring could instead be closer to the distal end of the sub-assembly, with the spring stretching rather than compressing. In this example, the proximal end of the spring is attached to the disc, and the distal end of the spring is attached to a component such as the housing, the rear housing, or the rotator.

Optionally, a dead coil (an inactive coil) could be provided at one or both ends of the spring. This can provide a larger surface area at the end of the spring for contact with adjacent components, which could be useful to avoid damage to adjacent components.

The disc is a ring in the depicted example, and is planar, but the shape of the disc could be varied. The disc is optional, and the feedback component could consist of a spring. Instead of being two separate components, the disc and the spring could be a single integral component. A second disc could be provided at the proximal end of the spring (i.e. between the spring and the syringe in this example). This could be beneficial in that it can provide a larger surface area for contact with the adjacent component (in this example the syringe), which can avoid placing a high pressure on a small area of the adjacent component (e.g. the syringe).

The particular shape of the depicted plunger rod is optional, though the tubular shape (specifically cylindrical in the depicted example) can be beneficial to support a plunger rod spring inside the plunger rod. An example of an optional feature is the planar area 79 at the proximal end of the plunger rod, which can be helpful for part orientation during assembly.

Instead of the proximal end of the arm 72 being attached to the rest of the plunger rod, the distal end of the arm 72 could instead be attached to the rest of the plunger rod, with the protrusion 74 at the proximal end of the arm instead. In general, the protrusion 74 could be spaced apart from the ends of the arm rather than being at the end of the arm.

The plunger rod arm 72 is optional—a protrusion 74 could extend directly from the rest of the plunger rod, with at least part of the plunger rod being made of a flexible material to allow the protrusion to disengage the feedback component. Instead of the protrusion moving out of the way to disengage the feedback component, the protrusion could be fixed, with the feedback component instead moving out of the way to disengage the feedback component from the plunger rod (for example by providing a flexible arm on the feedback component). The protrusion typically extends from an outer surface of the plunger rod. Optionally, the protrusion is a flange. Optionally, the protrusion is a proximal end of the plunger rod (this could be relevant in particular for providing feedback immediately before the injection starts so as to indicate the start of injection). The plunger rod is typically inside the housing, or at least partly inside the housing.

The devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Various modifications to the embodiments described are possible and will occur to those skilled in the art without departing from the invention which is defined by the following claims.

Some aspects of the invention are summarised in the following clauses.

1. A sub-assembly for a medicament delivery device (10), the sub-assembly extending along a longitudinal axis (12) in a longitudinal direction (13) from a proximal end (14) to a distal end (15), the sub-assembly comprising:
a plunger rod (70), the plunger rod (70) comprising a proximally facing surface (76) that faces towards the proximal end (14); and
a feedback component comprising a distally facing surface (82) facing towards the distal end (15), so that when the plunger rod (70) moves towards the proximal end (14) during use of the medicament delivery device (10), the distally facing surface (82) of the feedback component is engaged by the proximally facing surface (76) of the plunger rod, is subsequently moved towards the proximal end (14) by the plunger rod (70), and is finally disengaged from the proximally facing surface (76) of the plunger rod (70),
wherein the feedback component comprises a spring (90).

2. The sub-assembly of clause 1, wherein the plunger rod (70) comprises a protrusion (74), and the proximally facing surface (76) of the plunger rod (70) is on the protrusion (74).

3. The sub-assembly of clause 2, wherein the protrusion (74) extends from an outer surface of the plunger rod (70).

4. The sub-assembly of clause 2 or 3, wherein the plunger rod (70) comprises a flexible arm (72), and the protrusion (74) is attached to the flexible arm (72).

5. The sub-assembly of clause 4, wherein the protrusion (74) is attached to a distal end of the flexible arm (72).

6. The sub-assembly of clause 4 or 5, wherein the flexible arm (72) extends in the longitudinal direction (13), and wherein the flexible arm (72) is attached to the rest of the plunger rod (70) at a proximal end of the flexible arm (72).

7. The sub-assembly of any previous clause, wherein the proximally facing surface (76) of the plunger rod (70) is angled away from the longitudinal axis (12).

8. The sub-assembly of any previous clause, wherein the sub-assembly comprises a power source for moving the plunger rod (70) towards the proximal end (14).

9. The sub-assembly of clause 8, wherein the power source is a spring.

10. The sub-assembly of clause 9, wherein the spring is inside the plunger rod (70).

11. The sub-assembly of any of clauses 8 to 10, wherein the sub-assembly comprises a housing (30), and the plunger rod (70) is at least partly inside the housing (30).

12. The sub-assembly of any previous clause, wherein the sub-assembly comprises a proximally facing surface of a first further component, and wherein the distally facing surface (82) of the feedback component is adjacent to the proximally facing surface of the first further component.

13. The sub-assembly of clause 12, wherein the first further component is a housing (30).

14. The sub-assembly of clause 12, wherein the sub-assembly comprises a powerpack comprising the plunger rod (70), and the first further component is a component of the powerpack other than the plunger rod (70).

15. The sub-assembly of any of clauses 12 to 14, wherein the sub-assembly is arranged so that, when the distally facing surface of the feedback component disengages from the proximally facing surface of the plunger rod, the distally facing surface of the feedback component moves towards the distal end, thereby impacting the proximally facing surface of the first further component to provide feedback.

16. The sub-assembly of any previous clause, wherein the feedback component comprises a proximally facing surface facing towards the proximal end.

17. The sub-assembly of clause 16, wherein the sub-assembly comprises a distally facing surface of a second further component, and wherein the proximally facing surface of the feedback component is arranged adjacent to the distally facing surface of the second further component.

18. The sub-assembly of clause 17, wherein the second further component is a housing.

19. The sub-assembly of clause 17, wherein the second further component is a syringe carrier.

20. The sub-assembly of clause 17, wherein the second further component is a syringe.

21. The sub-assembly of any of clauses 17 to 20, wherein the proximally facing surface of the feedback component is arranged adjacent to the distally facing surface of the second further component so that the spring is compressed when the distally facing surface of the feedback component is moved towards the proximal end by the plunger rod.

22. The sub-assembly of any previous clause, wherein the feedback component provides a start click to provide an indication of the start of an injection and/or an end click to provide an indication of the end of an injection.

23. The sub-assembly of any previous clause, wherein the feedback component comprises a disc.

24. The sub-assembly of clause 23, wherein a proximally facing surface of the disc is adjacent to a distally facing surface of the spring.

25. A medicament delivery device comprising the sub-assembly of any previous clause.

26. The medicament delivery device of clause 25, wherein the medicament delivery device is an autoinjector.

1-15. (canceled)

16. A sub-assembly for a medicament delivery device, the sub-assembly extending along a longitudinal axis in a longitudinal direction from a proximal end to a distal end, the sub-assembly comprising:
a plunger rod, the plunger rod comprising a proximally facing surface that faces towards the proximal end; and
a feedback component comprising a distally facing surface facing towards the distal end, so that when the plunger rod moves towards the proximal end during use of the medicament delivery device, the distally facing surface of the feedback component is engaged by the proximally facing surface of the plunger rod, is subsequently moved towards the proximal end by the plunger rod, and is finally disengaged from the proximally facing surface of the plunger rod,
wherein the feedback component comprises a spring.

17. The sub-assembly of claim 16, wherein the plunger rod comprises a protrusion, and the proximally facing surface of the plunger rod is on the protrusion.

18. The sub-assembly of claim 17, wherein the plunger rod comprises a flexible arm, and the protrusion is attached to the flexible arm.

19. The sub-assembly of claim 18, wherein the protrusion is attached to a distal end of the flexible arm.

20. The sub-assembly of claim 18, wherein the flexible arm extends in the longitudinal direction, and wherein the flexible arm is attached to the rest of the plunger rod at a proximal end of the flexible arm.

21. The sub-assembly of claim 16, wherein the proximally facing surface of the plunger rod is angled away from the longitudinal axis.

22. The sub-assembly of claim 16, wherein the sub-assembly comprises a proximally facing surface of a first further component, and wherein the distally facing surface of the feedback component is adjacent to the proximally facing surface of the first further component.

23. The sub-assembly of claim 22, wherein the sub-assembly comprises a powerpack comprising the plunger rod, and the first further component is a component of the powerpack other than the plunger rod.

24. The sub-assembly of claim 22, wherein the first further component is a housing.

25. The sub-assembly of claim 16, wherein the feedback component comprises a proximally facing surface facing towards the proximal end.

26. The sub-assembly of claim 25, wherein the sub-assembly comprises a distally facing surface of a second further component, and wherein the proximally facing surface of the feedback component is arranged adjacent to the distally facing surface of the second further component.

27. The sub-assembly of claim 26, wherein the second further component is a housing, a syringe carrier or a syringe.

28. The sub-assembly of claim 16, wherein the feedback component provides a start click to provide an indication of the start of an injection and/or an end click to provide an indication of the end of an injection.

29. A medicament delivery device comprising the sub-assembly of claim 16.

30. The medicament delivery device of claim 29, wherein the medicament delivery device is an autoinjector.
